(19) United States
(12) Patent Application Publication
(10) Pub. No.: US 2025/0040962 A1
(43) Pub. Date: Feb. 6, 2025

(54) CLAMPING DEVICE FOR AN EXTERNAL FIXATOR

(71) Applicant: Hank Schiffers, Duisburg (DE)

(72) Inventor: Hank Schiffers, Duisburg (DE)

(21) Appl. No.: 18/636,182

(22) Filed: Apr. 15, 2024

(30) Foreign Application Priority Data
Oct. 13, 2021 (EU) EP21202393A

(51) Int. Cl. A61B 17/64

(52) U.S. Cl. CPC A61B 17/6466; A61B 17/6416

(57) ABSTRACT

A clamping device (100) for connecting pins (201) and/or bar elements (202) of an external fixator (200) is proposed, which has a first clamp (10) and a second clamp (20), wherein a tension element (31) connecting the first clamp (10) to the second clamp (20) is provided in a through hole (30) in the first clamp (10) and the second clamp (20), wherein the tension element (31) is mounted in the second clamp (20) and has a thread (33) engaging in a mating thread of a tightening means (40), wherein the tension element (31) is provided for bracing the first clamp (10) and the second clamp (20) by screwing of the thread (33) into the mating thread, wherein the clamping device (100) has a spring means (50) for pretensioning the first clamp (10) and the second clamp (20), wherein the tightening means (40) is arranged between the spring means (50) and the tension element (31).

The invention relates to a clamping device for connecting pins and/or bar elements of an external fixator for treating bone fractures. The clamping device has a first clamp and a second clamp, wherein a tension element connecting the first clamp to the second clamp is provided along an axial direction in a through hole in the first clamp and the second clamp. The tension element is mounted by a first end in the second clamp and has a thread lying opposite the first end and engaging in a mating thread of a tightening means. The tension element is intended for bracing the first clamp and the second clamp by screwing of the thread into the mating thread. The clamping device also has a spring means for pretensioning the first clamp and the second clamp.

Such clamps for use in an external fixator are known from the prior art. An external fixator is used for stabilizing and fixing bone fragments for emergency, preliminary treatment of a bone fracture and typically has a plurality of rod-shaped pins, clamping devices and at least one bar element.

The pins, which usually have a significantly smaller diameter than the bar elements, are provided to be anchored in the bone fragments in such a way that the pins partially protrude from the body of the injured person through the skin. For this purpose, the pins are in the form, for example, of screws, which are screwed into the bone fragments through the skin of the injured person.

The ends of the pins protruding from the skin are connected to each other via the bar elements by means of clamping devices and fixed relative to each other. In the case of simple bone fractures, it may be sufficient for only two bone fragments to be provided with pins and for the pins to be connected to each other by only one bar. If the bone fracture is more complicated, it is sometimes necessary for a multiplicity of bone fragments to have to be connected to one another. For this purpose, a multiplicity of pins are screwed into the bone fragments and connected to one another via a plurality of bar elements, if necessary also via joints.

One advantage of stabilizing a bone fracture with the aid of an external fixator is that the bone fragments do not have to be exposed. With the aid of the external fixator, the bone fragments can be provisionally stabilized so that the injured person is transportable. If there are further injuries, in particular life-threatening injuries, stabilizing the bone fracture with the external fixator allows the further injuries to be treated first when the bone fracture is provisionally stabilized.

The use of an external fixator after natural disasters or in war zones is particularly advantageous. This makes it possible to quickly transport injured persons with stabilized bone fractures out of the disaster area or the combat zone. If the patient is in a condition that allows the bone fracture to be operated on, the external fixator is removed and the bone fragments are fixed to one another and stabilized with internal aids, for example with nails, plates or screws.

When using an external fixator, the ease of use of the clamping devices is of particular importance. Especially in stressful situations and in the case of a particular rush to care for the injured person, it is desirable for the clamping devices to be able to be operated without tools and to be able to be applied and fixed in just a few simple steps.

A clamping device which has two clamps is known from document US 2012/089 142 A1. The two clamps are connected by a bolt, which has a thread at each of its two ends. The threads engage in nuts. If the nuts are screwed further onto the thread of the bolt, the clamps are braced and fixed in their position. Two springs are used to pretension the two clamps to move from a wide open position to a provisionally closed state.

It is the object of the present invention to provide an alternative clamping device, which is very easy to operate and requires a significantly reduced number of components compared to the prior art.

The object of the invention is achieved by a clamping device as claimed in claim 1.

In the case of the clamping device according to the invention, the tightening means is arranged between the spring means and the tension element. For the purposes of the present invention, “between” refers to the axial direction. The tension element is mounted by the first end in the second clamp. The second end, at which the thread of the tension element is arranged, lies opposite the first end in the axial direction. Preferably, it is provided that the first clamp and at least partially the second clamp are arranged between the first end and the second end of the tension element. Furthermore, it is preferably provided that the tightening means is arranged lying directly or indirectly against the first clamp. In particular, it is provided that the tightening means is arranged lying indirectly against a first clamp, i.e. that the tightening means is operatively connected to the first clamp by another component. If the thread of the tension element is screwed into the mating thread of the tightening means, the first clamp and the second clamp are compressed. The compression of the clamps causes the clamps to be braced, as a result of which bar elements or pins are held in the clamps.

If the clamps of the clamping device are not braced, the spring means of the clamping device causes pretensioning. The pretensioning makes it possible for bar elements or pins to be able to be held by the clamps without the clamps being tightly braced. This enables easy operation and adjustment of the clamping device in an advantageous way. The tension element, the tightening means and the spring means are arranged relative to each other such that, relative to the axial direction, the tightening means is arranged between the spring means and the tension element. This results in significantly more advantageous force flows during the pretensioning and bracing of the clamps, and therefore the maximum service life of the clamping device is advantageously extended.

Preferably, it is provided that the first clamp, the second clamp, the tension element and the tightening means are produced from a metal. It is conceivable for the tension element to be an elongate component which can be loaded under tension. It is conceivable for the tension element to have a chain or a cable. However, it is also conceivable for the tension element to have a post component.

Advantageous refinements and developments of the invention can be gathered from the dependent claims, and from the description with reference to the drawings.

According to a preferred embodiment of the present invention, it is provided that the first clamp has a first clamping jaw and a second clamping jaw and that the second clamp has a third clamping jaw and a fourth clamping jaw, wherein the first clamping jaw, the second clamping jaw, the third clamping jaw and the fourth clamping jaw each have a groove, wherein an open first receiving groove for receiving a bar element is formed by the grooves of the first clamping jaw and the second clamping jaw and an open second receiving groove for receiving a bar element of a is formed by the grooves of the third clamping jaw and the fourth clamping jaw, wherein the third clamping jaw and the fourth clamping jaw each have one, preferably two, further grooves, wherein at least one, preferably two, open further receiving grooves for receiving a pin are formed by the further grooves of the third clamping jaw and the fourth clamping jaw. For this purpose, it is preferably provided that the first receiving groove is provided at least in sections with first surface structures, which are designed to engage in a form-fitting manner in complementarily formed first further surface structures of a bar element, and/or the second receiving groove is preferably provided at least in sections with second surface structures, which are designed to engage in a form-fitting manner in complementarily formed first further surface structures of a bar element, and/or the further receiving groove or the further receiving grooves is or are preferably provided at least in sections with third surface structures, which are designed to engage in a form-fitting manner in complementarily formed second further surface structures of a pin.

This has an advantageous effect that pins or bar elements can be kept stable even if the clamps are not braced. Preferably, it is provided that the first clamping jaw, the second clamping jaw, the third clamping jaw and/or the fourth clamping jaw are formed integrally. Furthermore, it is preferably provided that the first clamping jaw, the second clamping jaw, the third clamping jaw and/or the fourth clamping jaw are produced from a metal, for example aluminum, steel or titanium. Preferably, the grooves of the first clamping jaw and the second clamping jaw are arranged lying opposite along the axial direction. Furthermore, the grooves of the second clamping jaw and the fourth clamping jaw are preferably arranged lying opposite along the axial direction. It is particularly preferably provided that the further receiving grooves are arranged running parallel to each other. Furthermore, it is particularly preferably provided that the further receiving grooves are arranged running at an angle and in particular perpendicular to the profile of the second receiving groove.

It is conceivable for the first and second clamping jaws to have intermeshing structures which are provided to prevent rotation of the first clamping jaw relative to the second clamping jaw. Furthermore, it is conceivable for the third and fourth clamping jaws to have intermeshing structures which are provided to prevent rotation of the third clamping jaw relative to the fourth clamping jaw. For this purpose it is conceivable for the intermeshing structures to have pegs which project along the axial direction and engage in depressions. This prevents the grooves from being displaced relative to each other. It is also conceivable for the first surface structures, the second surface structures and/or the third surface structures to have a serrated or ribbed structure.

The spring means causes pretensioning such that, in the pretensioned state of the clamping device, the diameter of the first receiving groove is smaller than the diameter of a bar element. Furthermore, the pretensioning of the spring means has the effect that, in the pretensioned state of the clamping device, the diameter of the second receiving groove is smaller than the diameter of a bar element and the diameter of the further receiving grooves is smaller than the diameter of a pin. For inserting a bar element or a pin into a receiving groove or a further receiving groove, it is provided that the bar element or the pin is pressed laterally, i.e. with a direction of movement which is arranged substantially orthogonally to the profile of the respective receiving groove or further receiving groove, into the receiving groove or further receiving groove. In this case, the diameter of the receiving groove or of the further receiving groove is increased briefly, counter to the pretensioning, before the diameter of the bar element or pin arranged in the receiving groove or further receiving groove is reduced again. The diameter of the receiving groove corresponds to the diameter of the bar element when said bar element is arranged in the receiving groove. The diameter of the further receiving groove corresponds to the diameter of the pin when said pin is arranged in the further receiving groove. The diameter within the meaning of the present invention is the maximum distance along the axial direction between the two grooves of a receiving groove or a further receiving groove.

Owing to the fact that only laterally open receiving grooves or laterally open further receiving grooves for receiving bar elements or pins are present, the use of housings, in particular the use of housings for receiving pins or for receiving bar elements, can be advantageously dispensed with. Preferably, therefore, the clamping device does not have a closed housing for receiving a bar element and/or a closed housing for receiving a pin.

Preferably, it is provided that rounded insertion regions for facilitating the insertion of the bar elements or pins are arranged on the open side of the receiving grooves. The rounded regions do not have rectilinear surfaces and allow for a much easier assembly of the fixator.

According to a further preferred embodiment of the present invention, it is provided that the clamping device has a handwheel, wherein the handwheel is in engagement with the tightening means in order to rotate the tightening means, wherein the tightening means is arranged lying against the handwheel along the axial direction. This makes it possible in an advantageous manner to rotate the tightening means by means of the handwheel and thus to screw the thread into the mating thread. Preferably, the handwheel is in a contrasting color to the remaining elements of the clamping device. This affords the advantage that it is immediately clear to the operator of the clamping device, even in a stressful situation, at which point the clamping device is to be operated. Preferably, it is further provided that the handwheel is manufactured from a metal. It is conceivable for the handwheel to have a lateral structure to facilitate operation. It is preferably provided that the handwheel is rotatable relative to the first clamp. Furthermore, it is preferably provided that the handwheel is arranged lying against the first clamp. In particular, it is provided that the first clamp is arranged between the second clamp and the handwheel. According to a preferred embodiment, it is provided that the handwheel is arranged partially enveloping the first clamp. For this purpose, it is preferably provided that the handwheel has a cantilever, which partially embraces the first clamping jaw.

Preferably, it is provided that the handwheel has a support element, which is arranged lying against the first clamping jaw. It is conceivable for the support element to project along the axial direction on the side of the handwheel facing the first clamping jaw. It is also conceivable for the support element to be arranged annularly around the tightening means. The tightening means is used to securely attach the handwheel to the first clamping jaw and transfers the spring force of the spring element for the pretensioning from the handwheel to the first clamping jaw.

According to another preferred embodiment of the present invention, it is provided that the tightening means is arranged between the handwheel and the first clamp, wherein the mating thread of the tightening means is arranged in a first further hole in the tightening means aligned with the through hole, wherein an engagement part of the tightening means is in form-fitting engagement with a receptacle of the handwheel. If the handwheel is rotated, the rotational movement advantageously causes a movement of the tension element along the axial direction. In particular, it is provided that the handwheel and the tightening means are two separate components.

For this purpose, it is provided, according to another preferred embodiment of the present invention, that the handwheel has a recess, wherein the spring means is arranged within the recess, wherein the spring means is arranged lying against a support point of the handwheel and against a pretensioning means, wherein the pretensioning means is in engagement with the tension element, wherein the spring means is arranged in such a way that a spring force of the spring means pulls the tension element along the axial direction in the direction of the handwheel. This advantageously brings about highly skillful guidance of the forces for the pretensioning. Preferably, it is provided that the spring means is arranged in such a way that the spring force of the spring means is arranged along the axial direction. It is also conceivable that the recess of the handwheel is arranged aligned with the hole. Preferably, it is provided that the spring means is completely arranged in the recess.

According to a further preferred embodiment of the present invention, it is provided that the pretensioning means has a locking screw, the further thread of which is in engagement with a further mating thread of the tension element, wherein, preferably, the spring means is arranged lying against a lower side of a screw head, which faces the support point, of the locking screw. The pretensioning means is thus provided to transmit the spring force of the spring means to the tension element in such a way that the tension element is arranged in a pretensioned state. For this purpose, the spring means in conjunction with the tension element ensures that the clamping jaws of the clamps are compressed between the bearing of the tension element in the second clamping jaw and the handwheel in such a way that bar elements or pins can be inserted into the corresponding receiving grooves and remain independently stable there. Preferably, it is provided that the screw head of the locking screw does not protrude beyond the handwheel in the axial direction. The locking screw also secures the clamping device against unintentional removal.

For transmitting the spring force of the spring means to the handwheel, it is preferably provided that the support point is arranged between the receptacle of the handwheel and the recess of the handwheel, wherein the support point is preferably formed integrally with the handwheel or wherein the support point has an annular disk which is inserted into an annular groove of the handwheel. Preferably, it is provided that the locking screw reaches through the support point. For this purpose, it is provided that the support point has an opening, which is penetrated by a part of the locking screw. In particular, it is provided that the screw head of the locking screw is dimensioned such that it does not fit through the opening. Furthermore, it is preferably provided that the spring means is arranged around the locking screw.

According to a further preferred embodiment of the present invention, it is provided that the receptacle has a profiled cross section, preferably a wavy and/or angular, particularly preferably square, hexagonal or octagonal, cross section, wherein the engagement part is arranged on an upper portion of the tightening means, wherein the upper portion has a profiled cross section, preferably a wavy and/or angular, particularly preferably square or hexagonal, cross section. This advantageously ensures that the handwheel is securely in engagement with the engagement part. Preferably, it is provided for this purpose that the tightening means has a lower portion, wherein the lower portion is arranged in the hole, wherein the lower portion preferably has a round cross section.

To provide a high degree of flexibility of the clamping device, it is preferably provided that the first clamp and the second clamp are mounted via a rotary joint so as to be rotatable with respect to each other about an axis of rotation and via a pivot joint so as to be pivotable with respect to each other about a pivot axis arranged perpendicularly to the axis of rotation, wherein the tension element is provided for fixing the first clamp relative to the second clamp with respect to the axis of rotation and the pivot axis by screwing of the thread into the mating thread. By this means, it is advantageously possible that the clamping device is fixed simultaneously with respect to the axis of rotation and with respect to the pivot axis and also the clamps are braced. For moving the clamping device from the pretensioned state, in which bar elements and pins can be inserted and the orientations of the clamps with respect to the axis of rotation and the pivot axis can be adjusted, into the braced state, in which the clamping device securely fixes pins and bar elements in a predetermined position, only the application of tension to the tension element, preferably in a single working step, is therefore necessary. This greatly simplifies the handling of the clamping device. The care of injured persons with an external fixator is facilitated, especially in stressful situations, and the time required for the care is reduced.

According to a further preferred embodiment of the present invention, it is provided that the rotary joint comprises at least two rotary joint components which are mounted rotatably with respect to each other about the axis of rotation and are adjacent to each other and which are lockable relative to each other in different angular positions by means of the tension element, wherein a first further spring means, which is designed to tension the rotary joint components away from each other in the axial direction, is arranged between the rotary joint components, wherein the rotary joint components have fourth surface structures which are arranged lying opposite each other, are formed in a complementary manner to each other and are designed to intermesh in a form-fitting manner in different angular positions, wherein the first further spring means is preferably formed integrally with one of the rotary joint components, wherein the first further spring means particularly preferably projects in the axial direction from the rotary joint component. It is thereby advantageously ensured that the clamps can be rotated against each other in a pretensioned state with respect to the axis of rotation and are fixed relative to each other in a braced state with respect to the axis of rotation. The first additional spring means spaces apart the rotary joint components of the rotary joint. Preferably, it is provided that the first clamp has one of the rotary joint components or that the second clamp has one of the rotary joint components.

The tension element is preferably guided centrally through the rotary joint components forming the rotary joint. For this purpose, the rotary joint components also have the hole. The first further spring means is preferably designed in the manner of a zigzag spring. Preferably, it is provided that the first further spring means has rings or ring elements which are arranged concentrically to each other and about the axial axis, and which are connected to each other via webs running in the axial direction. It is conceivable that the first further spring means is designed as a cylinder beam spring which can be subjected to a bending load or is subjected to a bending load and is therefore flexible. The first further spring means is preferably connected integrally to a rotary joint component. For this purpose, it is conceivable for the first further spring means to protrude from the rotary joint component in the axial direction. Preferably, it is provided for this purpose that the first further spring means has an annular element, which is connected integrally to the rotary joint component via webs. However, it is also conceivable for the first further spring means to be designed as a separate component. The rotary joint components are preferably disk-shaped and arranged lying opposite each other in the axial direction. Furthermore, it is conceivable for the fourth surface structures to have a serrated or ribbed structure. It is also conceivable for the fourth surface structures to be designed as circumferential gear rims.

According to a further preferred embodiment of the present invention, it is provided that the pivot joint comprises at least two pivot joint components which are mounted pivotably with respect to each other about the pivot axis and are adjacent to each other and which are lockable relative to each other in different angular positions by means of the tension element, wherein a second further spring means, which is designed to tension the pivot joint components away from each other in the axial direction, is arranged between the pivot joint components, wherein the pivot joint components have fifth surface structures which are arranged lying opposite each other and are formed in a mutually complementary manner and are designed to intermesh in a form-fitting manner in different angular positions.

By this means, it is advantageously brought about that the clamps can be pivoted in relation to one another in a pretensioned state with respect to the pivot axis and are fixed relative to one another in a braced state with respect to the pivot axis. The second further spring means spaces the pivot joint components of the pivot joint. Preferably, it is provided that the first clamp has one of the pivot joint components. For this purpose, it is provided in particular that the second clamp has a rotary joint component. Alternatively, it is preferably provided that the second clamp has one of the pivot joint components. For this purpose, it is provided in particular that the first clamp has a rotary joint component.

The tension element is preferably guided centrally through the pivot joint components forming the pivot joint. For this purpose, the pivot joint components also have the hole. The second further spring means is preferably designed as a leaf spring. In particular, it is provided that the leaf spring has a centrally arranged further opening through which the tension element is guided.

The pivot joint components are preferably disk-shaped and arranged in such a way that a pivot joint component with a substantially cylindrically convex side lies opposite a substantially cylindrically concave side of the other pivot joint component in the axial direction. Furthermore, it is conceivable for the fifth surface structures to have a serrated or ribbed structure.

It is conceivable for one pivot joint component to be connected integrally to a clamping jaw of a clamp and for the other pivot joint component to be connected integrally to a rotary joint component. For this purpose, it is preferably provided that the other rotary joint component, that is, the rotary joint component which is not connected integrally to the pivot joint component, is connected integrally to a clamping jaw of the other clamp, that is, the clamp which does not have a rotary joint component. In particular, it is provided that only the second further spring means and an integral structural element, which has a pivot joint component and a rotary joint component, is arranged between the first clamp and the second clamp.

According to a further preferred embodiment of the present invention, it is provided that the tension element is guided movably on the end side in a ball joint of the second clamp, in particular of the fourth clamping jaw, wherein the tension element has a head end, which is spherical at least in certain regions, wherein the head end is guided movably in a joint socket of the pivot joint. This enables a high degree of movability of the clamping device in a particularly advantageous manner. The ball joint makes it possible for the tension element to be able to follow a rotation about the axis of rotation and a pivoting movement about the pivot axis. In particular, it is thus advantageously brought about that the tension element can be moved in a 360° movement. The tension element is not reduced to a pivoting movement in a single plane. Furthermore, it is thus advantageously achieved that only very few components have to be used to realize the pivot joint. It is advantageously not necessary for an axle shaft to have to be arranged in an axle seat. Within the meaning of the present invention, guided and mounted are used synonymously.

In order to ensure that fixing and bracing of the components of the clamping device can be carried out simply and with little movement effort, it is preferably provided that the head end of the tension element has at least one fixing groove in which a protrusion projecting from the joint socket engages in such a way that rotation of the tension element about the axial direction is at least restricted. By restricting the rotation of the tension element about the axial direction, it is ensured that the tightening means can be rotated onto the tension element with the handwheel.

Further details, features and advantages of the invention emerge from the drawings and from the description below of preferred embodiments with reference to the drawings. The drawings illustrate merely exemplary embodiments of the invention that do not restrict the inventive concept.

In the drawings:

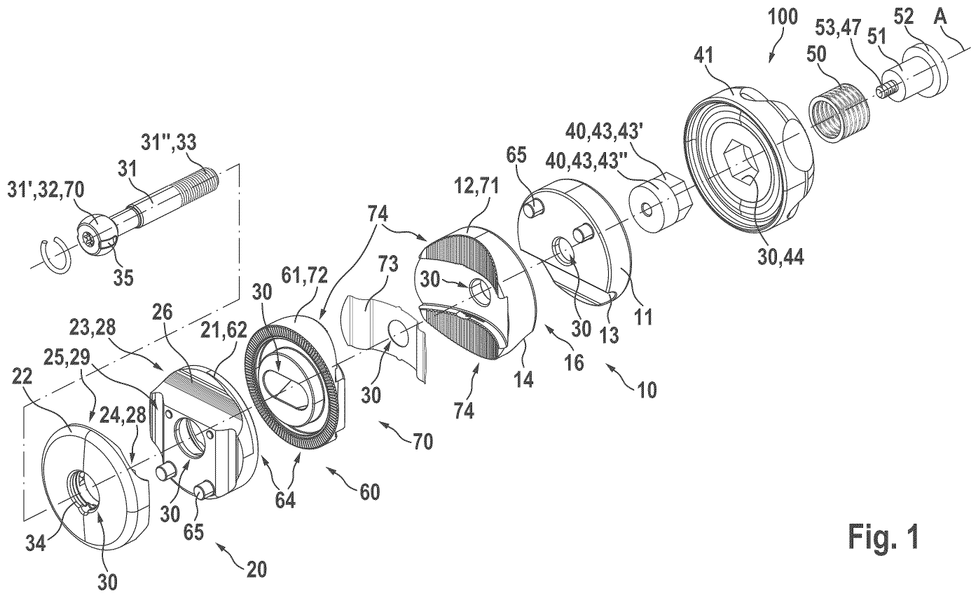

FIG. 1: shows a schematic exploded illustration of a clamping device according to an exemplary embodiment of the present invention,

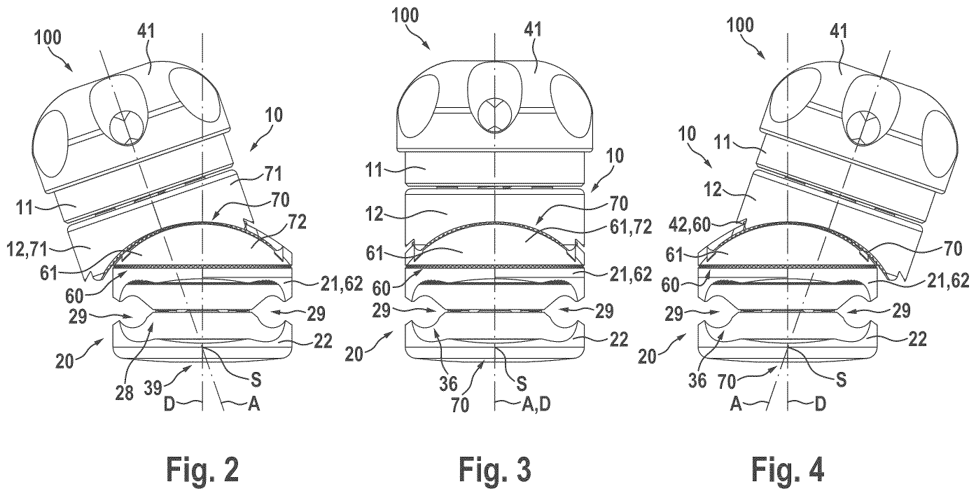

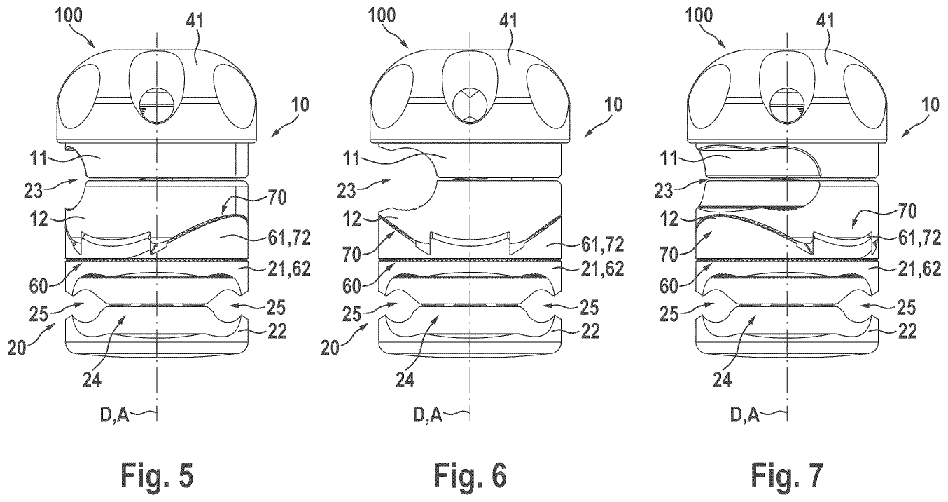

FIGS. 2-7: show schematic illustrations of a clamping device according to an exemplary embodiment of the present invention in different rotational or pivoting positions,

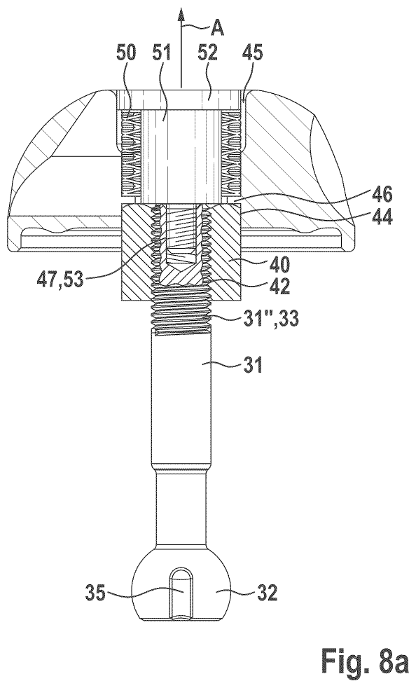

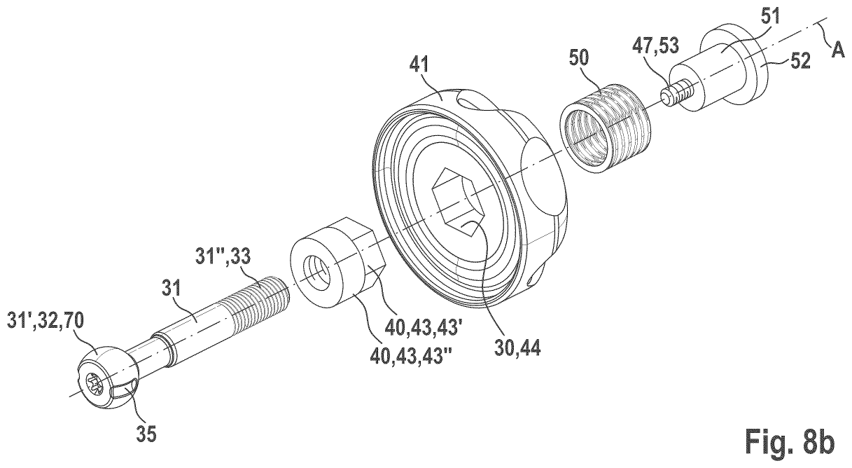

FIGS. 8a, b: show a schematic sectional illustration or a schematic exploded drawing of a detail of a clamping device according to an exemplary embodiment of the present invention,

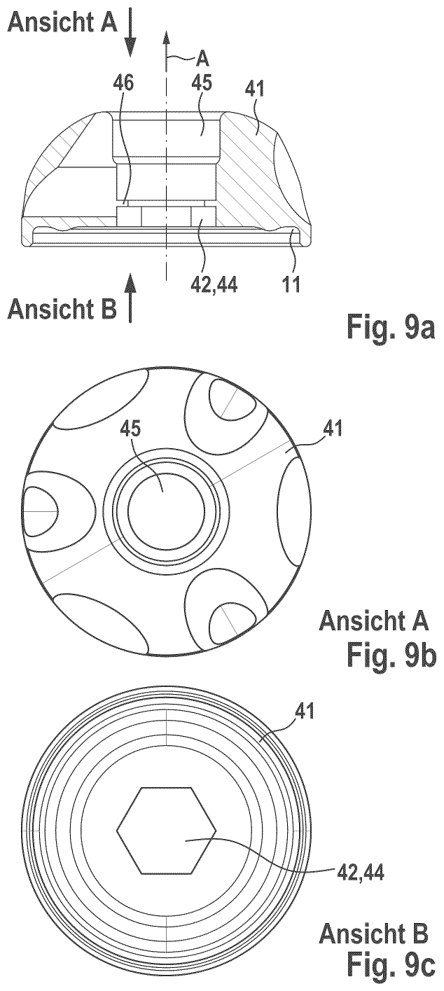

FIGS. 9a-c: show schematic illustrations of part of a clamping device according to an exemplary embodiment of the present invention,

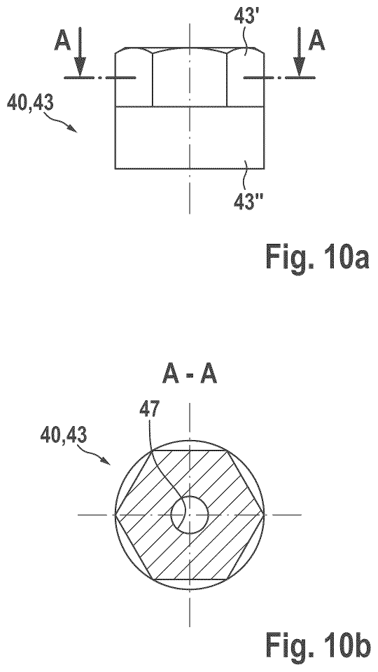

FIGS. 10a, b: show schematic illustrations of a tightening means of a clamping device according to an exemplary embodiment of the present invention,

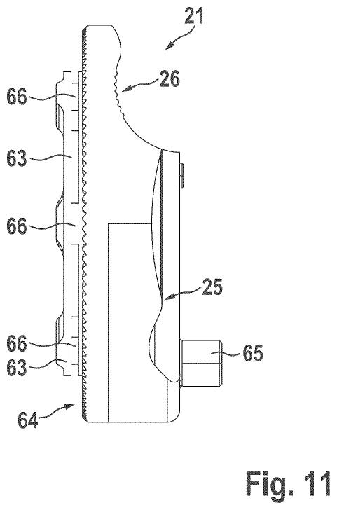

FIG. 11: shows a schematic illustration of the third clamping jaw of a clamping device according to an exemplary embodiment of the present invention,

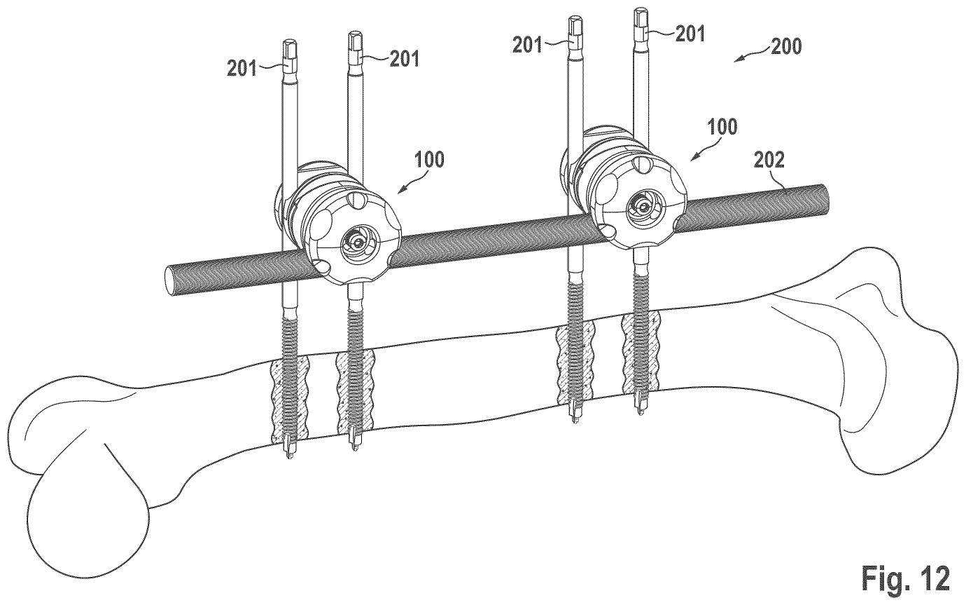

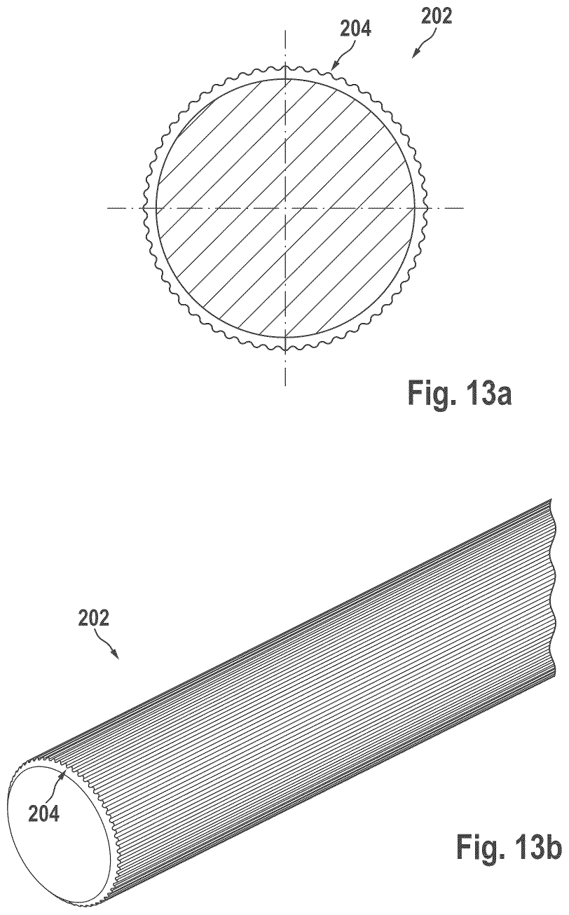

FIG. 12: shows an external fixator which has two clamping devices according to an exemplary embodiment of the present invention,

FIGS. 13a, b: show a bar element in a sectional illustration or in a perspective illustration for use with a clamping device according to an exemplary embodiment of the present invention.

FIG. 1 shows a clamping device 100 according to a preferred embodiment of the present invention in a schematic exploded illustration. The clamping device 100 is provided for connecting pins 201 and bar elements 202 of an external fixator 200 (see FIG. 12). An external fixator 200 is used, for example, for the preliminary care of bone fractures. For this purpose, pins 201 are anchored, for example screwed in, in bone fragments of the broken bone. At least one bar element 202 connects the pins 201 to each other and thus fixes the bone fragments relative to each other. For this purpose, the pins 201 and the at least one bar element 202 are connected to clamping devices 100.

A clamping device 100 has a first clamp 10 and a second clamp 20. The first clamp 10 is formed by a first clamping jaw 11 and a second clamping jaw 12. The first clamping jaw 11 and the second clamping jaw 12 are produced integrally preferably from a metal, particularly preferably from aluminum, steel or titanium. Both the first clamping jaw 11 and the second clamping jaw 12 each have a groove 13, 14, which are arranged lying opposite along the axial direction A in such a way that the grooves 13, 14 form a first open receiving groove 16 for receiving a bar element 202. The second clamp 20 is formed by a third clamping jaw 21 and a fourth clamping jaw 22. The third clamping jaw 21 and the fourth clamping jaw 22 are also produced integrally preferably from a metal, in particular from aluminum, steel or titanium. The third clamping jaw 21 and the fourth clamping jaw 22 have grooves 23, 24, which are arranged lying opposite along the axial direction A in such a way that they form a second open receiving groove 28 for receiving a bar element 202. The third clamping jaw 21 and fourth clamping jaw 22 furthermore have further grooves 25. The further grooves 25, here two further grooves 25, are arranged at a 90° angle to the second open receiving groove 28 and lie opposite each other along the axial direction A. The further grooves 25 form further receiving grooves 29, which are provided such that pins 201 are arranged in them.

To secure the clamping jaws 11, 12, 21, 22 against rotation and to ensure that the grooves 13, 14, 23, 24 and the further grooves 25 are arranged lying opposite one another in such a way that receiving grooves 16, 28 or further receiving grooves 29 are formed, intermeshing structures 65 are provided on the opposite surfaces of the clamping jaws 11, 12 of the first clamp 10 and the opposite surfaces of the clamping jaws 21, 22 of the second clamp 20. In the present case, the intermeshing structures 65 have protrusions, which engage in depressions (see in particular FIG. 11 in this regard).

If bar elements 202 are arranged in the receiving grooves 16, 28, a first surface structure 15 of the first clamp 10 or a second surface structure 26 of the second clamp 20 engages in a first further surface structure 204 of the bar elements 202 (see in particular FIG. 13, which shows a cross section of a bar element 202) and ensures a form-fitting fixing of the bar elements 202 in the receiving grooves 16, 28. Preferably, it is provided that the further receiving grooves 29 have third surface structures, which engage in second further surface structures of the pins 201 and fix the pins 201 in a form-fitting manner in the further receiving grooves 29.

In order to ensure a firm fit of the bar elements 202 or pins 201 in the clamping device 100, the clamping jaws 11, 12 of the first clamp 10 and the clamping jaws 21, 22 can be braced against each other.

The first clamp 10 and the second clamp 20 have a through hole 30 in which a tension element 31 is arranged along an axial direction A. The tension element 31 has an elongate, bolt-shaped basic body, at the first end 31′ of which an at least partially spherical head end 32 is arranged. A thread 33 is arranged at a second end 31″ which lies opposite the first end 31′. The tension element 31 is arranged with its spherical head end 32 in a joint socket 34 of the second clamp 20. In order to prevent rotation of the tension element 31 relative to the joint socket 34 about the axial direction A, a projecting protrusion (not visible here for perspective reasons) of the joint socket 34 engages in a fixing groove 35 of the spherical head end 32.

In order to be able to receive pins 201 and bar elements 202 in such a way that the bone fragments are fixed in an anatomically correct position with respect to each other, the clamping device 100 has to be provided to receive pins 201 and bar elements 202 with a different and situation-dependent spatial orientation. To adjust the position of the first receiving groove 16 relative to the second receiving groove 28 or to the further receiving grooves 29 (see FIGS. 2 to 7), the clamping device 100 has a rotary joint 60, which allows the first clamp 10 to rotate about an axis of rotation D relative to the second clamp 20.

For this purpose, the rotary joint 60 has rotary joint components 61, 62, which are mounted rotatably and are adjacent to each other. One of the rotary joint components 62 is formed by the third clamping jaw 21. The third clamping jaw 21 lies opposite the other rotary joint component 61 in the axial direction A. The two rotary joint components 61, 62 each have a fourth surface structure 64. The fourth surface structures 64 here are in the form of a toothed rim and are capable of intermeshing in a form-fitting manner in different angular positions of the rotary joint 60. In order to enable a rotational movement of the rotary joint 60, a first further spring means 63 ensures a spacing of the fourth surface structures 64. FIG. 11 shows the first further spring means 63 in detail. The first further spring means 63 is connected integrally to the third clamping jaw 21. It has webs 66 which jut out from the third clamping jaw 21 in the axial direction A and carry a circumferential ring 67. The circumferential ring 67 spaces the rotary joint components 61, 62 from each other such that the fourth surface structures 64 do not intermesh. If the rotary joint components 61, 62 are pushed together in the axial direction A, the first further spring means 63 bends and allows a form-fitting connection of the fourth surface structures 64. The clamping device 100 is thus secured against further rotational movements about the axis of rotation D.

In the embodiment shown of the clamping device, a rotary joint component 62 is realized by the third clamping jaw 21.

For further adjustment of the position of the first receiving groove 16 relative to the second receiving groove 28 or the further receiving grooves 29, the clamping device 100 furthermore has a pivot joint 70 for pivoting the first clamp 10 relative to the second clamp 20 about a pivot axis S, which is arranged perpendicular to the axis of rotation D. The pivot joint 70 is formed by pivot joint components 71, 72 and a second further spring means 73. A pivot joint component 71 has a concavely cylindrical surface which is arranged lying opposite a convexly cylindrical surface of the other pivot joint component 72. The center points of the concavely or convexly cylindrical shape of the surfaces lie substantially in the center of the spherical head end 32 of the tension element 31. A fifth surface structure 74 is arranged on each of the concavely cylindrical and convexly cylindrical surfaces of the pivot joint components 71, 72, said fifth surface structures being designed to intermesh in different angular positions of the pivot joint 70. In order to enable a pivoting movement of the pivot joint 70, the second further spring means 73 ensures a spacing of the fifth surface structures 74. The second further spring means 73 is provided here as a leaf spring, which has a further opening 75 in the form of an elongate hole through which the tension element 31 reaches. If the pivot joint components 71, 72 are pressed against each other counter to the spring force of the second further spring means 73, the fifth surface structures 74 intermesh in a form-fitting manner and fix the first clamp 10 and the second clamp 20 relative to each other with respect to the pivot axis S.

In the embodiment shown of the clamping device 100, a pivot joint component 71 is realized by the second clamping jaw 12. Furthermore, the other pivot joint component 72 is connected integrally to a rotary joint component 61, which is not part of the third clamping jaw 21.

To fix the first clamp 10 relative to the second clamp 20 with respect to the axis of rotation D and the pivot axis S and to brace the first clamp 10 and the second clamp 20, the thread 33 of the tension element 31 engages in a mating thread of a tightening means 40 (see in particular FIG. 8). The tightening means 40 has an engagement part 43, which is in form-fitting engagement with a receptacle 44 of a handwheel 41 of the clamping device 100. If the handwheel 41 is rotated, the tightening means 40 rotates at the same time and the thread 33 of the tension element 31 is screwed into or out of the mating thread of the tightening means 40. The first clamping jaw 11, the second clamping jaw 12 with one of the pivot joint components 71, the other pivot joint component 72 with one of the rotary joint components 61, and the third clamping jaw 21 with the other rotary joint component 62 are arranged between the fourth clamping jaw 22 and the handwheel 41. If the thread 33 of the tension element 31 is screwed into the mating thread of the tightening means 40, the tension element 31 pulls the clamping jaws 11, 12, 21, 22, the rotary joint 60 and the pivot joint 70 together such that the surface structures 15, 26, 64, 74 and 204 intermesh in a form-fitting manner. This means that the rotary joint 60 and the pivot joint 70 are fixed with a single rotary movement on the handwheel 41 and the clamps 10, 20 are braced.

In order to ensure a form-fitting engagement of the engagement part 43 with the handwheel 41, it is provided that an upper portion 43′ of the engagement part 43 is arranged in a receptacle 44 of the handwheel 41. The receptacle 44 of the handwheel 41 is arranged aligned with the hole 30. The receptacle 44 of the handwheel 41 has a profiling. This profiling can be wavy, for example similar to a Torx carrying profile (Torx is a registered trademark) or a Stardrive carrying profile (Stardrive is a registered trademark). For example, hexalobular socket profiles, octalobular socket profiles or decalobular socket profiles are conceivable here. However, all other polygonal shapes, for example a square, hexagonal or octagonal cross section, are also conceivable. This can be seen in particular in FIG. 9c, which shows a view of the handwheel 41 from below. The upper portion 43′ of the engagement part 43 is formed correspondingly to the profiling of the receptacle 44. FIG. 10a shows a tightening means 40. The profiled upper portion 43′ is clearly visible. FIG. 10b shows a section through the upper portion 43′, which has an octagonal cross section here.

The tightening means 40 protrudes with a lower portion 43″ into the hole 30. The support point 46 is preferably connected integrally to the handwheel 41. However, it is also conceivable for the support point 46 to be formed by a circular ring-shaped disk, which is embedded in an annular groove of the handwheel 41.

The clamping device 100 has a spring means 50, which is provided to pretension the clamping jaws 11, 12, 21, 22 of the first clamp 10 and the second clamp 20. For this purpose, the spring means 50 is arranged in a recess 45 of the handwheel 41 and lies against the support point 46 and against a pretensioning means 51 in such a way that the spring force of the spring means 50 pushes the pretensioning means 51 away from the support point 46 along the axial direction A. The recess 45 is arranged aligned with the receptacle 44 and has a round cross section (see FIG. 9b, which shows the handwheel 41 from above). The pretensioning means 51 is provided as a locking screw which, by way of a further thread 53, is in engagement with a further mating thread 47 of the tension element 31. The tightening means 40 is arranged here between the tension element 31 and the spring means 50, and therefore the support point 46 is arranged between the tightening means 40 and the spring means 50. The spring means 50 is supported on a screw head 52 of the pretensioning means 50. If the pretensioning means 51 is moved along the axial direction A by the spring means 50, the tension element 31 is also pulled along the axial direction A, which leads to pretensioning of the clamping jaws 11, 12, 21 and 22.

### LIST OF REFERENCE SIGNS

10 first clamp
11 first clamping jaw
12 second clamping jaw
13 groove
14 groove
15 first surface structure
16 first receiving groove
20 second clamp
21 third clamping jaw
22 fourth clamping jaw
23 groove
24 groove
25 further groove
26 second surface structure
28 second receiving groove
29 further receiving groove
30 through hole
31 tension element
31 first end
31″ second end
32 head end
33 thread
34 joint socket
35 fixing groove
40 tightening means
41 handwheel
42 first further hole
43 engagement part
43′ upper portion
43″ lower portion in
44 receptacle
45 recess
46 support point
50 spring means
51 pretensioning means
52 screw head
60 rotary joint
61 rotary joint component
62 rotary joint component
63 first further spring means
64 fourth surface structure
70 pivot joint
71 pivot joint component
72 pivot joint component
73 second further spring means
74 fifth surface structure
75 further opening
100 clamping device
200 external fixator
201 pin
202 bar element
204 first further surface structure
A axial direction
D axis of rotation
S pivot axis

1. A clamping device (100) for connecting pins (201) and/or bar elements (202) of an external fixator (200) for treating bone fractures, having a first clamp (10) and a second clamp (20), wherein a tension element (31) connecting the first clamp (10) to the second clamp (20) is provided along an axial direction (A) in a through hole (30) in the first clamp (10) and the second clamp (20), wherein the tension element (31) is mounted by a first end (31′) in the second clamp (20) and, at a second end (31″) lying opposite the first end (31′), has a thread (33) engaging in a mating thread of a tightening means (40), wherein the tension element (31) is provided for bracing the first clamp (10) and the second clamp (20) by screwing of the thread (33) into the mating thread, wherein the clamping device (100) has a spring means (50) for pretensioning the first clamp (10) and the second clamp (20), characterized in that the tightening means (40) is arranged between the spring means (50) and the tension element (31).

2. The clamping device (100) as claimed in claim 1, characterized in that the first clamp (10) has a first clamping jaw (11) and a second clamping jaw (12) and in that the second clamp (20) has a third clamping jaw (21) and a fourth clamping jaw (22), wherein the first clamping jaw (11), the second clamping jaw (12), the third clamping jaw (21) and the fourth clamping jaw (22) each have a groove (13, 14, 23, 24), wherein an open first receiving groove (16) for receiving a bar element (202) is formed by the grooves (13, 14) of the first clamping jaw (11) and the second clamping jaw (12) and an open second receiving groove (28) for receiving a bar element (202) of a is formed by the grooves (22, 24) of the third clamping jaw (21) and the fourth clamping jaw (22), wherein the third clamping jaw (21) and the fourth clamping jaw (22) each have one, preferably two, further grooves (25), wherein at least one, preferably two, open further receiving grooves (29) for receiving a pin (201) are formed by the further grooves (25) of the third clamping jaw (21) and the fourth clamping jaw (22), wherein the first receiving groove (16) is preferably provided at least in sections with first surface structures (15), which are designed to engage in a form-fitting manner in complementarily formed first further surface structures (204) of a bar element (202), and/or the second receiving groove (28) is preferably provided at least in sections with second surface structures (26), which are designed to engage in a form-fitting manner in complementarily formed first further surface structures (204) of a bar element (202), and/or the further receiving groove (29) or the further receiving grooves (29) is or are preferably provided at least in sections with third surface structures, which are designed to engage in a form-fitting manner in complementarily formed second further surface structures (203) of a pin (201).

3. The clamping device (100) as claimed in claim 2, characterized in that the clamping device (100) has a handwheel (41), wherein the handwheel (41) is in engagement with the tightening means (40) in order to rotate the tightening means (40), wherein the tightening means (40) is arranged lying against the handwheel (41) along the axial direction (A).

4. The clamping device (100) as claimed in claim 3, characterized in that the tightening means (40) is arranged between the handwheel (41) and the first clamp (10), wherein the mating thread of the tightening means (40) is arranged in a first further hole (42) in the tightening means (40) aligned with the through hole (30), wherein an engagement part (43) of the tightening means (40) is in form-fitting engagement with a receptacle (44) of the handwheel (41).

5. The clamping device (100) as claimed in claim 4, characterized in that the handwheel (41) has a recess (45), wherein the spring means (50) is arranged within the recess (45), wherein the spring means (50) is arranged lying against a support point (46) of the handwheel (41) and against a pretensioning means (51), wherein the pretensioning means (51) is in engagement with the tension element (31), wherein the spring means (50) is arranged in such a way that a spring force of the spring means (50) pulls the tension element (31) along the axial direction (A) in the direction of the handwheel (41).

6. The clamping device (100) as claimed in claim 5, characterized in that the recess (45) is arranged aligned with the hole (30).

7. The clamping device (100) as claimed in claim 6, characterized in that the pretensioning means (51) has a locking screw, the further thread (53) of which is in engagement with a further mating thread (47) of the tension element (31), wherein, preferably, the spring means (50) is arranged lying against a lower side of a screw head (52), which faces the support point (46), of the locking screw.

8. The clamping device (100) as claimed in 7, characterized in that the support point (46) is arranged between the receptacle (44) of the handwheel (41) and the recess (45) of the handwheel (41), wherein the support point (46) is preferably formed integrally with the handwheel (41) or wherein the support point (46) has an annular disk which is inserted into an annular groove of the handwheel (41).

9. The clamping device (100) as claimed in 8, characterized in that the receptacle (44) has a profiled cross section, preferably a wavy and/or angular, particularly preferably square, hexagonal or octagonal, cross section, wherein the engagement part (43) is arranged on an upper portion (43′) of the tightening means (40), wherein the upper portion (43′) has a profiled cross section, preferably a wavy and/or angular, particularly preferably square or hexagonal, cross section.

10. The clamping device (100) as claimed in claim 9, characterized in that the tightening means has a lower portion (43″), wherein the lower portion (43″) is arranged in the hole (30), wherein the lower portion (43″) preferably has a round cross section.

11. The clamping device (100) as claimed in claim 10, characterized in that the first clamp (10) and the second clamp (20) are mounted via a rotary joint (60) so as to be rotatable with respect to each other about an axis of rotation (D) and via a pivot joint (70) so as to be pivotable with respect to each other about a pivot axis(S) arranged perpendicularly to the axis of rotation (D), wherein the tension element (31) is provided for fixing the first clamp (10) relative to the second clamp (20) with respect to the axis of rotation (D) and the pivot axis(S) by screwing of the thread (33) into the mating thread.

12. The clamping device (100) as claimed in claim 11, characterized in that the rotary joint (60) comprises at least two rotary joint components (61, 62) which are mounted rotatably with respect to each other about the axis of rotation (D) and are adjacent to each other and which are lockable relative to each other in different angular positions by means of the tension element (31), wherein a first further spring means (63), which is designed to tension the rotary joint components (61, 62) away from each other in the axial direction (A), is arranged between the rotary joint components (61, 62), wherein the rotary joint components (61, 62) have fourth surface structures (64) which are arranged lying opposite each other, are formed in a complementary manner to each other and are designed to intermesh in a form-fitting manner in different angular positions, wherein the first further spring means (63) is preferably formed integrally with one of the rotary joint components (61, 62), wherein the first further spring means (63) particularly preferably projects in the axial direction (A) from the rotary joint component (61, 62).

13. The clamping device (100) as claimed in claim 12, characterized in that the pivot joint (70) comprises at least two pivot joint components (71, 72) which are mounted pivotably with respect to each other about the pivot axis(S) and are adjacent to each other and which are lockable relative to each other in different angular positions by means of the tension element (31), wherein a second further spring means (73), which is designed to tension the pivot joint components (71, 72) away from each other in the axial direction (A), is arranged between the pivot joint components (71, 72), wherein the pivot joint components (71, 72) have fifth surface structures (74) which are arranged lying opposite each other and are formed in a mutually complementary manner and are designed to intermesh in a form-fitting manner in different angular positions.

14. The clamping device (100) as claimed in claim 13, characterized in that the tension element (31) is guided movably on the end side in a ball joint of the second clamp (20), in particular of the fourth clamping jaw (22), wherein the tension element (31) has a head end (32), which is spherical at least in certain regions, wherein the head end (32) is guided movably in a joint socket (34) of the pivot joint (70).

15. The clamping device (100) as claimed in claim 14, characterized in that the head end (32) of the tension element (31) has at least one fixing groove (35) in which a protrusion projecting from the joint socket (34) engages in such a way that rotation of the tension element (31) about the axial direction (A) is at least restricted.
